(19) United States
(12) Patent Application Publication
(10) Pub. No.: US 2006/0222687 A1
(43) Pub. Date: Oct. 5, 2006

(54) Topical anesthetic

(76) Inventors: Stephen G. Carter, Andover, MA (US); Zhen Zhu, Tewksbury, MA (US); Kanu Patel, Derry, NH (US)

Correspondence Address: NIELDS & LEMACK, 176 EAST MAIN STREET, SUITE 7, WESTBORO, MA, 01581, US

(21) Appl. No.: 11/096,394

(22) Filed: Apr. 1, 2005

(51) Int. Cl. A61K 9/70; A61M 31/00

(52) U.S. Cl. 424/443; 604/500

(57) ABSTRACT

A formulation and method for the preparation of topical anesthetics formulations, containing one or more anesthetics, in a delivery vehicle designed to promote penetration of the anesthetics into the skin and underlying tissue and to facilitate the speed of action and prolonged response. The stratum corneum layer of the skin at the application site is at least partially removed by an abrading substance, such as pumice, preferably incorporated into the formulation, or by a step that uses a mechanical abrasion of the outer layer of skin, such as a rotating or vibrating brush. The enhanced penetration into the live skin tissue is achieved by removing the outer layer of stratum corneum skin tissue by including an abrading substance in a formulation containing the anesthetics, then rubbing the formulation into the skin, or the use of a brush-like device to assist in the removal of the skin layer.

### BACKGROUND OF THE INVENTION

Prior art describes that there are topical anesthetics that may be used to give relief from skin irritations and localized tissue pain. These anesthetics include but are not limited to lidocaine, benzocaine, and tetracaine. The application of these agents, using simple emulsion-based formulations, results in a fast acting, but typically not a strong, anesthetic effect. The object of this invention is to create a similar but unique type of treatment, which is used topically to serve as a pretreatment to several medical and cosmetic procedures, such as laser treatments for the treatment of acne and scars, and hair removal.

Currently used topical treatments require the use of the agents to be applied to the skin, and left on the skin for a period of almost one hour with results of anesthesia to the pain associated with the procedures being minimum. In this invention, we describe the development of a series of formulations designed to assist in the penetration of the topically applied anesthetics into the skin tissue, with the result of a stronger and longer acting and faster onset of anesthetic effect.

A key barrier to the successful demonstration of topical anesthesia is the outer layer of skin. The present invention encompasses the removal of this layer in order to enhance the activity of the anesthetics by promoting penetration of the active agent. For example, the inclusion of abrading substances within the formulation of anesthetics physically removes the stratum corneum layer of skin upon rubbing, assisting the penetrating formulation components in entering into the skin tissue and promoting an anesthetic effect. Alternatively, the stratum corneum layer of skin can be removed by other means, such as the mechanical force of a rotating or vibrating brush, such as that used to apply the anesthetic formulations.

### SUMMARY OF THE INVENTION

Topical anesthetics are used in everyday applications to treat minor irritations and discomforts. This list of potential anesthetics is long and may be used alone or in combination with more than one anesthetic agent to elicit different kinetics of the reaction or response. The problem noted with most if not all of these agents is that they take some time to demonstrate the response. In addition, the response on the skin and in the treated tissue underneath the skin is typically not strong enough to allow for the administration of minor or non-invasive medical or cosmetic procedures, which may cause some minor discomfort. This invention uses commonly available topical anesthetics, in combination with chemical penetration enhancers, to assist and enhance the penetration of the anesthetics, in a manner sufficient to allow the anesthetics to induce a state of anesthesia in the local tissue sufficient to permit the conduct of procedures to the skin, without significant discomfort. This action is promoted or permitted by the use of substances or actions to partially or completely remove the stratum corneum layer of skin in the vicinity of the application site, or to exfoliate the top layer of skin or the hyperkeratotic tissue formed there. Removal of the stratum corneum layer is performed by using microabrading substances such as but not limited to pumice, and/or by using a mechanical rubbing apparatus prior to application of the topical formulation, during application, or both.

It is therefore an object of the present invention to provide a topical anesthetic formulation comprising an effective amount of at least one anesthetic and a skin-abrading agent such as pumice, lufa, and/or aluminum oxide.

It is a further object of the present invention to provide a method of anesthetizing the skin of a patient with a topical anesthetic formulation comprising an effective amount of an anesthetic and a skin-abrading agent, wherein the method comprises rubbing the formulation on the skin so as to cause the skin-abrading agent to at least partially remove the stratum corneum layer to enhance the penetration of the formulation into the skin tissue.

It is yet a further object of the present invention to provide a method of anesthetizing the skin of a patient with a topical anesthetic formulation comprising an effective amount of an anesthetic, wherein the method comprising mechanically abrading the skin at an application site to at least partially remove the stratum corneum layer, and applying the topical anesthetic formulation to the application site and allowing said formulation to penetrate that site.

It is a still further object of the invention to at least partially remove the stratum corneum layer of skin at a site and topically apply an anesthetic to that site as a pretreatment to one or more medical or cosmetic procedures, such as laser treatments for the treatment of acne and scars, and hair removal.

### DETAILED DESCRIPTION OF THE INVENTION

The current invention describes the development of topical formulations containing one or more anesthetics to be presented topically to the subject in a manner that would promote maximum penetration into the skin tissue of a mammal, preferably a human. The process used to ensure maximum penetration into the live skin tissue uses the inclusion of chemical grade abrasion substances, which will physically remove the outer layer of skin, giving direct access to the skin tissue to receive anesthesia. Alternatively, the anesthetic and the abrading substance could be formulated in separate formulations and applied in sequence.

Once the stratum corneum layer has been removed, the lipids or lipid-like chemicals employed in the formulations assist in the penetration of the anesthetics into the skin. Enhanced penetration of the anesthetics agents into the skin tissue promote interaction of these agents with the nerve cells and fibers, for the purpose of creating maximum loss of pain and sensation in a short period of time after the application and also remaining for a long lasting effect.

Suitable abrading substances include pumice, preferably in an amount effective for at least partially removing the stratum corneum layer of the skin, the partial removal being sufficient to enhance the penetration of the anesthetic agent compared to such penetration had no skin been removed. The abrading substance may be used as part of the formulation in concentrations ranging from about 0.1 to about 8% by weight, depending on the tissue and also on the composition of the remainder of the formulation. Alternatively or in addition, other suitable abrasive substances include lufa and/or aluminum oxide, again in an amount effective for at least partially removing stratum corneum layer, preferably from about 0.1 to about 10% by weight, more preferably from about 0.25 to about 8% by weight. Partial removal of the stratum corneum layer is removal of a sufficient amount of this layer to enhance the penetration of the anesthetic agent compared to such penetration had no skin been removed. The pumice, lufa and aluminum oxide can be used alone or in any combination.

The anesthetics may be used as a single agent or in combination with other anesthetics to elicit a stronger or different type of effect. Examples of suitable anesthetics include but are not limited to benzocaine in a concentration range of about 0-18% w/w; lidocaine in a concentration range of about 0-18% w/w; and tetracaine, in a concentration range of about 0-18% w/w, or combinations thereof. These anesthetics are formulated in combination with substances designed to promote penetration into the outer layer of the skin, including but not limited to natural oils rich in gamma linoleic acid such as hemp oil (about 0-5%), propylene glycol (about 0-30%) and menthol (about 0-10%), which assist in the movement of the anesthetic materials from the skin surface, through the outer layer of the skin.

In an alternative embodiment of the present invention, the abrading substance may be excluded from the formulation and instead, the layer of skin is partially or completely removed by mechanical abrading, such as with a mechanical rotating or vibrating brush. The brush is used to agitate and then at least partially remove the stratum corneum layer. This procedure can be carried out prior to the application of the topical anesthetic, or can be carried out concurrently with the application of the topical anesthetic. Indeed, the mechanical tool used to abrade can be the same tool used to apply the formulation. The formulation also can include the abrading substance if desired.

The delivery complex preferably also includes chemicals that function as penetration enhancers to assist in the transport of the anesthetics from the skin surface, into the dermal layer of the skin. Suitable enhancers include by example only but are not limited to: individual fatty acids, fatty acid esters, polyols, amides, various anionic, cationic and nonionic surfactants such as but not limited to sodium laurate and sodium lauryl sulfate, phospholipids, cholesterol and cholesterol derivatives, m-pyrrole, dimethyl acetamide, limonene, sphingolipids, ceramides, terpenes, alkanones, menthol, various organic acids, such as but not limited to salicylic acid, citric and succininc acid, prostaglandins, decyl methyl sulfoxide, urea, sulfoxide alcohols, plant extract oils. Suitable fatty acids include by example but are not limited to: linoleic acids, linolenic acids, oleic acids, stearic acids, and myristic acids. Phospholipids include by example but are not limited to: phosphatidylcholine, phosphatidylethanolamine, and phosphatidylserine. Plant extract oils include peanut oil, hemp, barag, olive oil, sunflower oil, soybean oil, monoi oil and macadamia oil, with olive oil being preferred. Suitable amounts of plant extract oil or plant extract oil/alcohol mix in the delivery complex range from about 1% to about 66% by weight, more preferably from about 10% to about 33.3% by weight. This component may serve exclusively as the penetrating agent or it may also, in addition, serve another function to the delivery complex such as vasodilation, as the active drug agent, or binding of the delivery complex. One or more penetrating agents or chemically modified penetrating agents may be used in varying quantities or ratios with respect to the other component parts in the drug delivery complex at any one time. The penetrating agent molecule may also serve in any of the other critical functions for the delivery system, including that of active drug molecule, vasodilator, and/or polymer.

The delivery complex may optionally include a polymer or a formulation stabilizer molecule. This substance is designed to be compatible with the composition of the remainder of the chemicals in the formulation and typically to be an inert functional component that will be degraded in the skin or designed to not penetrate into the skin. Examples of suitable polymers include but are not limited to: carbopol, hydroxyethylcellulose, u-care polymer, and water-soluble gums (e.g., agar, arabic, carob, CMC, carrageenans, ghatti, guar, karaya, kadaya, locust bean, tragacanth, and xanthan gums). The polymer should be used in an amount ranging from about 1% to about 20% by weight, most preferably an amount equal to the amount of active ingredient used. Exceptions may be made to this statement if the polymer is serving both the function of the polymer as well as the active drug molecule or agent or in the function of vasodilator and/or in the function of the penetration enhancer. The polymer may serve exclusively as the polymer or it may also, in addition, serve another function to the delivery complex such as vasodilation, penetration, or as the active drug agent of the delivery complex.

The preparation of the abrasive-containing, topical anesthetic vehicle may be achieved through standard formulation processes, depending on the chemical characteristics of the specific components. Typically, a hydrogel or aqueous or non-aqueous emulsion is prepared by first preparing a mixture of the water-soluble chemicals and mixing until homogenous, then blending, through a slow addition of the non-aqueous phase of the mixture until homogeneity is achieved. Homogenization steps, either during or as a final stage in the process, are optimal for a stable and homogeneous preparation.

The therapeutic dose of the formulation of the present invention may vary depending on the desired effect and also on the use of the specific anesthetics. The dose and the frequency of the dosing may also vary according to the age, body weight and response of the individual patient. In general, 1-5 grams of the formulation per application is desired. The amount should be applied as if a moisturizing cream were to be applied, covering the area, and then rubbing into the skin tissue until it is absorbed. The manual rubbing action of the formulation containing the abrasive substance should be performed for a time sufficient to disrupt the intact stratum corneum layer and also sufficient to absorb the anesthetics into the exposed epidermis. Rubbing for about 30 seconds has been found to be effective for this purpose.

### FORMULATION EXAMPLE 1

| | Weight by % |
|---|---|
| Benzocaine | 12 |
| Lidocaine | 5 |
| Tetracaine | 1.0 |
| Clove oil | 1 |
| Frescolate ML | 1 |
| Isopropanol | 27 |
| Xanthan gum | 1 |
| Pumice | 4 |
| Kaoline | 8 |
| Water | 40 |

### FORMULATION EXAMPLE 2

| | Weight by % |
|---|---|
| Benzocaine | 12 |
| Lidocaine | 5 |
| Tetracaine | 1.0 |
| Aluminum oxide | 4.0 |
| Clove oil | 1 |
| Frescolate ML | 2 |
| Isopropanol | 35 |
| Xanthan gum | 1 |
| Water | 39 |

### FORMULATION EXAMPLE 3

| | Weight by % |
|---|---|
| Benzocaine | 12 |
| Lidocaine | 5 |
| Tetracaine | 5 |
| Clove oil | 1 |
| Lufa | 8 |
| Menthol | 1 |
| Propylene glycol | 4 |
| Ethanol | 23 |
| Sweet almond oil | 8 |
| Beeswax | 10 |
| Microcrystalline wax | 4 |
| Water | 19 |

### FORMULATION EXAMPLE 4

| | Weight by % |
|---|---|
| Benzocaine | 12 |
| Lidocaine | 5 |
| Tetracaine | 1 |
| Clove oil | 1 |
| Pumice | 6 |
| Menthol | 1 |
| Propylene glycol | 7 |
| Dimethylacetamide | 10 |
| Ethanol | 37 |
| Ucare JR-30M | 0.5 |
| Cellosize QP 5200 | 0.5 |
| Water | 19 |

### APPLICATION EXAMPLE 1

This is an example of the treatment of local facial skin tissue for the purpose of anesthesia prior to and during a dermatologic procedure. The subject washes their face with warm water and soap, dries the face then over an area such as either a cheek or chin or forehead and approximately 1 gram of formula from Example 1 (see above) is applied to the skin, with a finger rubbing the sample into the skin. The sample is typically rubbed in a circular pattern for approximately 30 seconds. The remainder of the formula is absorbed or left on the skin to become absorbed for a period of 5-10 minutes. After this period of time, the remainder of the lotion is wiped off the face with a clean cloth. The treatment (e.g., laser treatment for acne) may be initiated after the 5-10 minutes anesthesia incubation period and it may be expected to remain numb for up to 60 minutes. A typical procedure that may follow the application of the topical anesthetic is a laser treatment for acne and other dermal scars or inflammation, a procedure that causes considerable discomfort to the patient if it is performed without any form of anesthesia. Additionally, other minor dermatological procedures that have previously caused the patient discomfort, including dermal abrasion and scraping may also be procedural candidates for a pretreatment with one of the topical anesthesia formulations described in this application.

### APPLICATION EXAMPLE 2

The subject washes their face with warm water and soap, dries the face then over an area such as either a cheek or chin or forehead, Approximately 1 gram of a skin preparation lotion is applied to the skin with a finger rubbing the sample into the skin in a circular pattern for approximately 30 seconds. The skin preparation lotion will contain variations of the following prototype composition, 8% pumice, 2% cetyl alcohol, 2% lipomulse, 10% propylene glycol, 82% deionized water. The remainder of the skin preparation lotion is wiped off the skin, followed by a 1 gram application of the anesthetic lotion, similar to formula examples 1-4 above. The 1 gram of anesthetic formula is rubbed into the skin with a finger for approximately 30 seconds, then left to absorb for another 5-10 minutes. At this time, the skin should be numb and the procedure may be performed over the next 30-45 minutes.

### APPLICATION EXAMPLE 3

The subject washes their face with warm water and soap, dries the face then over an area such as either a cheek or chin or forehead approximately 1 gram of the formulation of example 1 (see above) is applied to the skin, using a mechanical application device similar to an electric, circular pattern toothbrush. The anesthetic lotion is applied to the skin for a period of approximately 30 seconds with the device, then the remainder of the formula is absorbed or left on the skin to become absorbed for a period of 5-10 minutes. The remaining material is wiped off the face with a clean cloth. The treatment (e.g., laser treatment for acne) may be initiated after the 5-10 minutes anesthesia incubation period and it may be expected to remain numb for up to 60 minutes.

Other examples of use applications and procedures where the use of a topically applied anesthetic may be useful include but are not limited to minor medical procedures, conducted in a doctor's office or on an out-patient basis, treatment of skin irritation resulting from abrasion or insect bites or stings, or other pretreatments before a dermatologic procedure that may cause discomfort from either pain, heat or pressure sensations.

What is claimed is:

1. A topical anesthetic formulation, comprising an anesthetic-effective amount of at least one anesthetic and a skin-abrading agent.

2. The topical anesthetic formulation of claim 1, wherein said skin-abrading agent is selected from the group consisting of pumice, lufa, aluminum oxide and combinations thereof.

3. The topical anesthetic formulation of claim 1, wherein said anesthetic is selected from the group consisting of lidocaine, benzocaine, tetracaine and combinations thereof.

4. The topical anesthetic formulation of claim 2, wherein said anesthetic is selected from the group consisting of lidocaine, benzocaine, tetracaine and combinations thereof.

5. The topical anesthetic formulation of claim 1, further comprising a penetration enhancer.

6. The topical anesthetic formulation of claim 1, further comprising a vasodilator.

7. The topical anesthetic formulation of claim 1, wherein said skin abrading agent is present in an amount effective for at least partially removing the stratum corneum layer at the site of application of the formulation.

8. A method of anesthetizing the skin of a patient with a topical anesthetic formulation comprising an anesthetic-effective amount of an anesthetic and a skin-abrading agent, said method comprising applying said formulation on the skin so as to cause said skin-abrading agent to at least partially remove the stratum corneum layer, and allowing said formulation to penetrate the site of application.

9. The method of claim 8, wherein said skin-abrading agent is selected from the group consisting of pumice, lufa, aluminum oxide and combinations thereof.

10. The method of claim 8, wherein said anesthetic is selected from the group consisting of lidocaine, benzocaine, tetracaine and combinations thereof.

11. The method of claim 9, wherein said anesthetic is selected from the group consisting of lidocaine, benzocaine, tetracaine and combinations thereof.

12. The method of claim 8, wherein said topical anesthetic formulation further comprising a penetration enhancer.

13. The method of claim 8, wherein said topical anesthetic formulation further comprises a vasodilator.

14. The method of claim 8, further comprising carrying out a dermatological medical procedure at said site of application.

15. The method of claim 14, wherein said procedure comprises laser application.

16. A method of anesthetizing the skin of a patient with a topical anesthetic formulation comprising an anesthetic-effective amount of an anesthetic, said method comprising mechanically abrading the skin at an application site to at least partially remove the stratum corneum layer, and applying said topical anesthetic formulation to said application site and allowing said formulation to penetrate said site.

17. The method of claim 16, wherein said mechanical abrasion is carried out prior to or during the application of said formulation.

18. The method of claim 16, further comprising carrying out a dermatological medical procedure at said site of application.

19. The method of claim 18, wherein said procedure comprises laser application.
